(19) United States
(12) Patent Application Publication
(10) Pub. No.: US 2014/0234922 A1
(43) Pub. Date: Aug. 21, 2014

(54) SYSTEM AND METHOD FOR THE PRODUCTION OF ALKYL ESTERS

(71) Applicant: VIESEL SKUNK WORKS, LLC, Stuart, FL (US)

(72) Inventors: Graham Towerton, Dickinson, TX (US); Stuart Lamb, Stuart, FL (US)

(73) Assignee: VIESEL SKUNK WORKS, LLC, Stuart, FL (US)

(21) Appl. No.: 14/186,356

(22) Filed: Feb. 21, 2014

Related U.S. Application Data
(60) Provisional application No. 61/767,391, filed on Feb. 21, 2013.

(51) Int. Cl. C12P 7/62

(52) U.S. Cl. CPC C12P 7/62
USPC 435/135

(57) ABSTRACT

A method of producing alkyl esters including processing a high free fatty acid feedstock including a mixture of triglycerides and free fatty acids to remove water and solids to create a processed feedstock. The processed feedstock is introduced and mixed into a reaction vessel, the reaction vessel includes water, at least one enzyme, and alcohol. The reacted contents are separated into a glycerin phase and an alkyl ester phase. The alkyl ester phase is treated with a primary alcohol and a flocculant.

### CROSS-REFERENCE TO RELATED APPLICATION

This application is related to and claims priority to U.S. Provisional Patent Application Ser. No. 61/767,391, filed Feb. 21, 2013, entitled SYSTEM AND METHOD FOR THE PRODUCTION OF ALKYL ESTERS, the entirety of which is incorporated herein by reference.

### STATEMENT REGARDING FEDERALLY SPONSORED RESEARCH OR DEVELOPMENT

n/a

### FIELD OF THE INVENTION

The present invention relates to a method and system for the production of alkyl esters, or biodiesel, and more particularly, to a system and method for the production of biodiesel.

### BACKGROUND OF THE INVENTION

Diesel fuel made from crude petroleum has been used for nearly a century to fuel diesel engines. Originally, these engines were designed to run on vegetable oil. Biodiesel, or alkyl esters made from fatty acids, are also suitable for use in diesel engines. Additionally, biodiesel has a wide range of other valuable uses for manufacturing specialty chemicals used in the industry, agriculture, and janitorial cleaning products.

Alkyl esters may be made from vegetable oils or fats that contain triglycerides and free fatty acids (FFAs). FFAs are present in oils which have been degraded by heat, chemicals, or water contamination, which degradation may cause the fatty acids of the oil to be liberated from the glycerin molecule in the triglyceride structure. Most alkyl ester production processes are quite limited in the amount of FFA that can be present in the oil. Typically, without pre-treatment of the oil to remove FFA, many production processes for biodiesel can only accept feed stock oils of <4% FFA. Processes that make use of pre-treatment, such as acid esterification, can accept 20-40% FFA; however, this creates significant cost for use acids, alcohols, and energy, as well as additional safety risks for plant operation.

Low FFA biodiesel processes typically operate by exposing the triglycerides to an alcohol (for example, methanol) in the presence of a base catalyst, such as sodium or potassium hydroxide, in a trans-esterification reaction. High FFA biodiesel processes will first reduce the FFA by exposing the triglyceride/FFA mixture to an alcohol (e.g., methanol) in the presence of an acid (e.g., sulfuric acid) in an esterification reaction which converts the FFA to alkyl esters, leaving the majority of triglycerides for conversion in a subsequent trans-esterification reaction. It is generally known that the reaction rates for these reactions can be increased by using higher temperatures, higher pressures, or excess amounts of alcohol and catalyst.

Many methods have been used to perform these reactions, using batch, semi-batch, or continuous reaction processes. Batch reactions were first developed for alkyl ester production in the 1930s and 1940s and were found to involve too much time and energy to produce adequate yields. Several semi-batch processes and several continuous processes have been developed to successfully shorten the reaction time, but these processes involve high temperatures, high pressures, and still require use of chemical catalysts. Very few of these processes have been developed to accommodate high FFA levels. Because of the high temperatures, pressures, and chemicals involved, the equipment used for the processes must be designed accordingly with suitable high-strength materials that are resistant to corrosion. This creates significant costs for the biodiesel producer in the form of engineering design, fabrication, and construction costs, typically making use of stainless steel or other suitable but expensive alloys.

Successful increases in reaction kinetics have been achieved by using alcohol in high ratios relative to the stoichiometric reaction requirements. Acid esterification reactions typically require a 20:1 molar ratio of methanol:fatty acid in order to ensure good yields and conversion with short reaction times. Alkaline trans-esterification reactions typically require a 6:1 molar ratio of alcohol to triglycerides for the same purposes of increasing reaction kinetics. Use of such high ratios of alcohol generally requires significant considerations for managing process safety as well as the need to recover, purify, and recycle significant quantities of alcohol.

All of these processes previously described typically require large amounts of energy. The production of biodiesel is aimed at replacing the use of petroleum fuels, thereby reducing the amount of carbon dioxide released to the atmosphere due to combustion of fossil fuels. It is therefore of significance to reduce energy consumption in the biodiesel manufacturing process, thereby maximizing the environmental gains derived from the production of this fuel. The United States Environmental Protection Agency (EPA) rewards this energy efficiency by assigning an equivalency value (EV) based on the percentage of greenhouse gas emissions reduction achieved by the production process for making renewable fuels. Many of the earlier developments in biofuel production have relatively low equivalency values, thereby reducing the perceived value of the fuels produced from these processes, defined by net energy gained relative to the energy expended in its production.

Few novel technologies have been developed that allow for the use of a single reaction process for conversion of triglycerides and FFA to alkyl esters, without the use of a two-stage or three-stage reaction process. This has limited the exploration into the use of low cost raw materials that may contain high levels of FFA, such as brown grease, trap grease, tall oil, highly degraded animal fats, or other waste fats and oils that may be recovered, for example, from municipal and industrial water treatment facilities. For these feed stocks, which may include up to 100% FFA, enzymatic catalysts may be used. However, despite advances in the use of enzymatic catalysts, several drawbacks still exist that prevent the full commercial exploitation of this technology. For instance, enzymatic catalysis may be conducted using either immobilized enzymes (that is, enzymes that are bound to a solid substrate) or liquid enzyme (that is, fully mobile enzymes in a liquid solution). Immobilized enzymes provide the advantage of allowing the enzymes to be held within the reaction vessel so that they are not lost with the reaction fluids that leave the reactor. However, this creates additional costs for creating the immobilized enzymes and for the design and operation of the reaction system to allow the retention of enzymes within the reactor. Further, the immobilized enzymes must be replaced at certain intervals once their activity deteriorates to a level that prevents full conversion of triglycerides and FFA to alkyl esters.

Even when using liquid enzymes, the recovery and re-use of the enzymes is important in order to keep costs low. Currently, enzymes are recovered by recycling the entire heavy phase into subsequent reactions. This results in an increasing volume of heavy phase, which must be recycled and which reduces the volume of feedstock that can be processed in any subsequent reaction. After a certain number of reaction cycles, the volume of recycled heavy phase becomes so great that the reaction process is rendered cost ineffective. Additionally, the liquid enzymes become so dispersed within the increased heavy phase that the reaction rates become severely limited.

Additionally, very few advances have been made to develop commercially economical technologies for improving the processing of a light phase, such as separation and recovery of alkyl esters from the reaction products that may contain glycerin, water, soaps, unreacted triglycerides, FFA, catalyst salts, alcohol, and particulate matter. The biodiesel industry employs numerous methods for purifying biodiesel and glycerin, including distillation, ion exchange, physical and chemical adsorption, water washing, and filtration. However, these methods generally require an additional large capital investment for all of these process operations and additional energy and water consumption.

For example, the light phase from the enzymatic reaction process, which may be a mixture of alcohol and alkyl esters, may contain FFA levels between approximately 1% and approximately 5% of the light phase volume, but will typically be between approximately 2.5% and approximately 3% of the light phase volume. This FFA must be neutralized and removed in order to meet suitable specifications for use as a fuel (for example, a biodiesel) or base oil for lubricants and specialty chemicals production. Currently known methods for the removal of FFA include the introduction of a dilute solution of sodium hydroxide or other alkaline chemicals (known as “caustic washing”) to convert the FFA to an insoluble soap which can then be separated from the alkyl esters by gravity or by known mechanical methods; the use of immobilized enzymatic technology to convert the FFA to alkyl esters; and distillation of the alkyl esters. However, these methods require the use of harmful chemicals and high levels of energy, involve the production of a waste byproduct, and/or increase the cost of biodiesel production.

As a result of the wide range of raw materials available for alkyl ester production, and the equally wide range of reaction processes and post-reaction purification processes, no single biodiesel production plant design has yet been achieved on a commercial scale that fully optimizes energy usage, makes use of low-cost capital equipment, and maximizes the use of low-cost raw materials.

It is therefore desirable to provide a method and system for the production of alkyl esters, or biodiesel, that decreases or eliminates the need to use harmful chemicals, high temperature, and high pressure. It is also desirable to provide a method and system for the production of alkyl esters, or biodiesel, that allows for the effective removal of FFA with minimal waste byproduct production and minimal final treatment required to ensure the product biodiesel meets required quality specifications.

### SUMMARY OF THE INVENTION

The present invention advantageously provides for a method of producing alkyl esters. The method includes processing a high free fatty acid feedstock including a mixture of triglycerides and free fatty acids to remove water and solids to create a processed feedstock. The processed feedstock is introduced and mixed into a reaction vessel, the reaction vessel includes water, at least one enzyme, and alcohol. The reacted contents are separated into a glycerin phase and an alkyl ester phase. The alkyl ester phase is treated with a primary alcohol and a flocculant.

In another embodiment, the method includes processing a high free fatty acid feedstock including a mixture of triglycerides and free fatty acids to remove water and solids to create a processed feedstock. The processed feedstock is introduced and mixed into a reaction vessel, the reaction vessel including water, at least one enzyme, and alcohol. The reacted content is separated into a glycerin phase and an alkyl ester phase. The alkyl ester phase is washed with an aqueous base. The alkyl ester phase is treated with a methanol and a polyacrylamide cationic polymer.

In yet another embodiment, the method includes processing a high free fatty acid feedstock including a mixture of triglycerides and free fatty acids to remove water and solids to create a processed feedstock. The processed feedstock is introduced and mixed into a reaction vessel, the reaction vessel including water, at least one enzyme, and alcohol. The reacted contents are separated into a glycerin phase and an alkyl ester phase. A portion of the at least one enzyme is recycled from the glycerin phase. Water and the least one alcohol is removed from the alkyl ester phase. Free fatty acids in the alkyl ester phase are converted into alkyl esters. The alkyl ester phase is washed with an aqueous base. The alkyl ester phase is treated with a methanol and a polyacrylamide cationic polymer, wherein the alkyl ester phase has a weight, and the percentage of methanol used to treat the alky ester phase is approximately 2 percent of the weight of the alkyl ester phase.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a flow chart illustrating an alkyl ester production process in accordance with the principles of the present invention; and

FIG. 2 is a schematic view of an alkyl ester production process in accordance with the principles of the present invention.

### DETAILED DESCRIPTION OF THE INVENTION

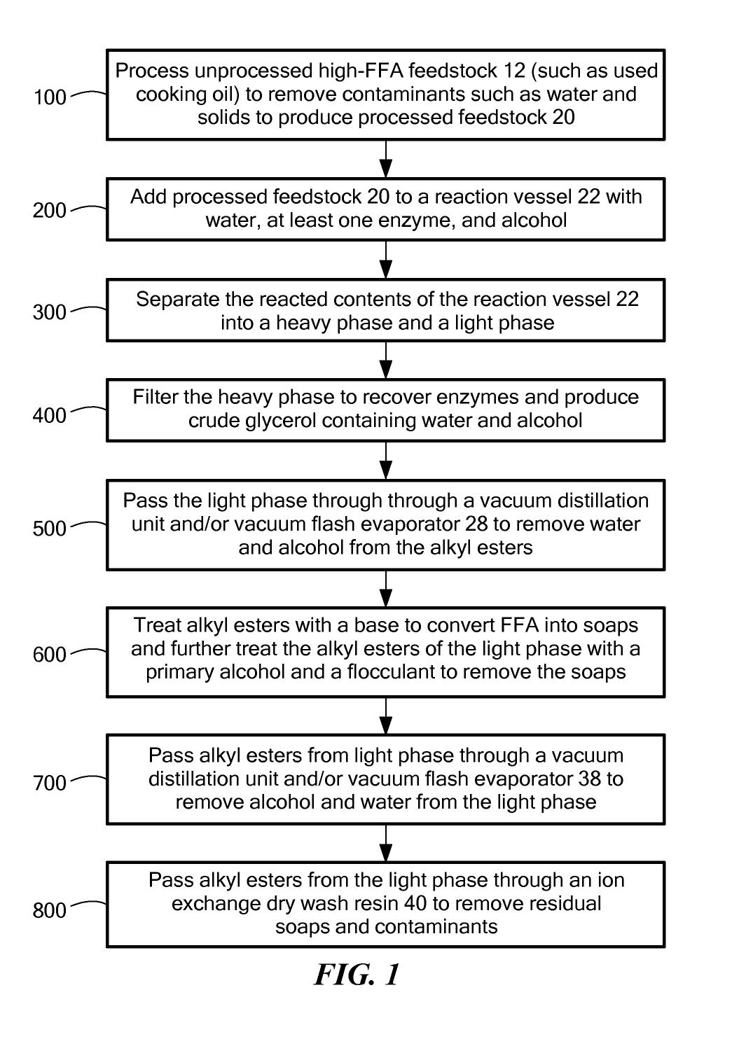

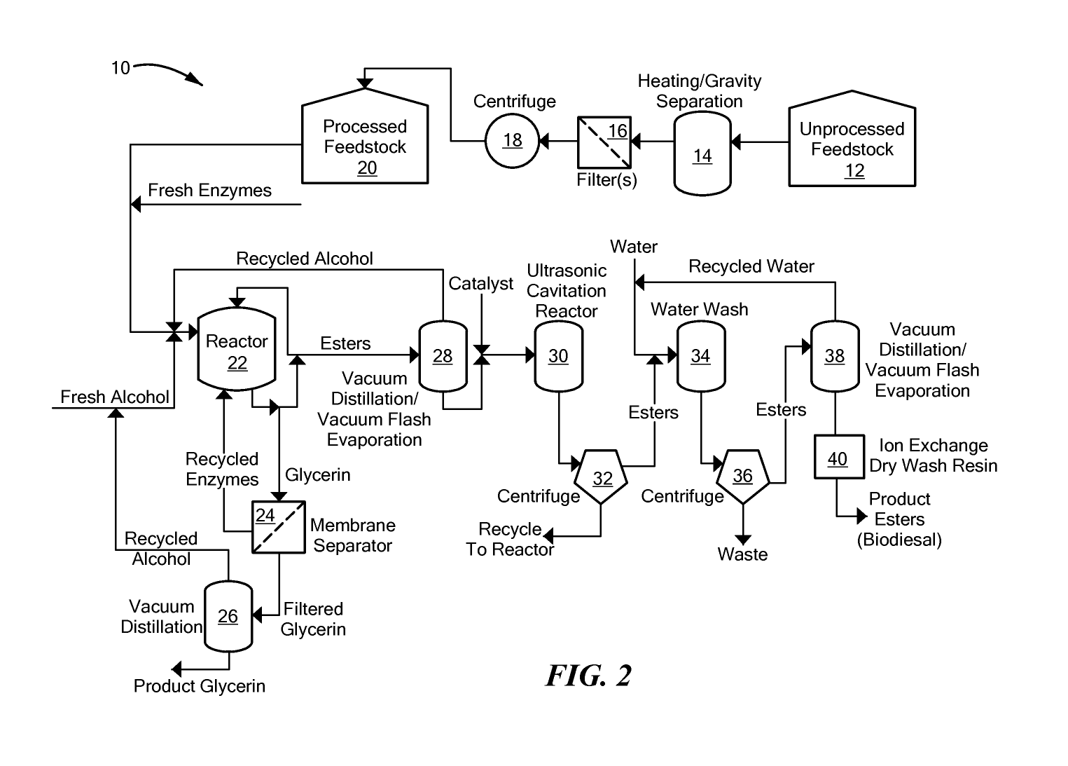

Referring now to the drawings in which like reference designators refer to like elements, there is shown in FIGS. 1 and 2 an exemplary method and system for the production of alkyl esters constructed in accordance with the principles of the present invention. Unlike presently known transesterification methods, the present invention provides a method and system 10 for the production of biodiesel from high-FFA feedstock (for example, used cooking oil) without the need for harmful chemicals, high temperature, and high pressure. The present invention also provides a method and system 10 for the production of alkyl esters, or biodiesel, that allows for the effective removal of FFA with minimal waste byproduct production and minimal final treatment required to ensure the product biodiesel meets required quality specifications.

In the first step 100, unprocessed feedstock 12, for example, used cooking oil, may be processed to remove contaminants such as water and solids. As a non-limiting example, this may be achieved through, for example, heating and gravity separation 14 of water and solids, followed by several stages of filtration 16 starting with, for example, a filter size of approximately 400 microns, which may be reduced to an approximately 1-micron nominal filter size. After filtration, the feedstock 12 may be centrifuged 18 in a disk-stack centrifuge for final removal of fine sediment and free water.

At this point in the process, the feedstock 12 may be referred to as processed feedstock 20. In the second step 200, the processed feedstock 20 may be added to an enzymatic biodiesel reaction vessel or reactor 22 in combination with a requisite amount of water, alcohol, and at least one enzyme. The alcohol may be fresh or re-used alcohol (for example, methanol or ethanol). The at least one enzyme may be, for example, a liquid enzyme such as lipase. While there are several types of lipase that may be used, in an exemplary embodiment, lipases containing Candida antarctica Lipase B (CALB) may be used. For example, enzyme catalyst usage may be 1 wt % based on the cooking oil weight. An example of alcohol usage is the use of methanol at approximately 13-15 wt % based on cooking oil weight with methanol added continuously for approximately 10-14 hours from the start of the reaction. The processed feedstock 20, water, alcohol, and at least one enzyme may then be circulated and mixed within the biodiesel reactor 22 for approximately 16-24 hours.

In the third step 300, a sample of the reaction fluids within the biodiesel reactor 22 may be tested to confirm completion of the reaction. The reaction fluids may then be allowed to remain in the biodiesel reactor 22 to settle within the reaction, thereby allowing a heavy phase to separate from a light phase. Alternatively, a centrifuge or coalescer may be used to achieve this separation. A sample of the reaction fluids in the biodiesel reaction may be tested to confirm reaction completion.

In the fourth step 400, the recovered heavy phase, which may also be referred to as the glycerin phase, may be removed from the biodiesel reactor 22 to a storage tank (not shown). The heavy phase may include glycerin (glycerol), alcohol, water, and enzymes. The heavy phase (glycerin phase) may be retained in the reactor 22 for subsequent reactions or it may be passed through, for example, a membrane separator 24 to filter at least one enzyme from the heavy phase. The membrane separator 24 may recover the enzymes in a concentrated form and may produce a crude glycerol containing water and alcohol. The recovered enzymes may be stored in a tank or other container (not shown) prior to reuse in subsequent reactions. Further, the storage tank may be chilled to reduce enzyme activity losses that may occur prior to reuse of the recovered enzymes. Additionally, the heavy phase may be purified, in which separated enzymes may be recycled back into the reactor 22. The filtered glycerin, which at this state may be referred to as crude glycerin, may then undergo processing such as vacuum distillation 26. The distillation process may separate the alcohol portion of the heavy phase, which may be recycled back into the reactor 22. The distillation process may also separate glycerol, also referred to as a product glycerin portion, which may be collected, stored, and/or used or sold for other applications. The separated light phase, which may also be referred to as the ester phase or alkyl ester phase, may include alkyl esters, water, and alcohol. In the fifth step 500, water and alcohol may be removed from the light phase (ester phase). This process may include vacuum distillation and/or vacuum flash evaporation 28.

In the sixth step 600, the alkyl esters remaining in the light phase may then be processed through an ultrasonic cavitation reactor 30 in combination with alcohol and catalyst to allow for the conversion of the free fatty acid to fatty acid alkyl esters. As a non-limiting example, the ultrasonic reactor may operate at approximately 20 kHz. The light phase may then be centrifuged 32. The light phase may then go through a caustic wash process 34 for the treatment and removal of free fatty acids. In the caustic wash process, the light phase may be processed using an aqueous base, such as a caustic soda, to convert free fatty acids to soaps. Most of these soaps may be removed by gravity separation, but some soaps may remain suspended in the light phase, which will become biodiesel. The full removal of soaps may be achieved by the use of chemical flocculants and centrifugation 36. A non-limiting example of flocculants that may be used to remove soaps from the light phase may include a polyacrylamide cationic polymer. The use of chemical flocculant may be enhanced by the addition of a primary alcohol. For example, methanol may be included in the treatment of the light phase, but other primary alcohols, such as propanol, butanol, ethanol, and/or any C1-C4 alcohols may be used. In an exemplary configuration, methanol may be combined with the flocculant at approximately 2 wt % to approximately 2.5 wt % based on the weight of the light phase. The resultant removal of soap with the use of flocculant and methanol may typically reduce soap levels to less than approximately 500 ppm, enabling efficient use of a centrifuge for the removal of soaps to approximately 50 ppm. This may allow the alkyl esters to proceed through the final processing steps.

In the seventh step 700, the alkyl esters may be processed using a vacuum distillation and/or vacuum flash evaporation process 38 to further remove alcohol and water from the light phase. In the eighth step 800, the alkyl esters may be passed through an ion exchange dry wash resin 40 that removes residual soaps and contaminants (such as salts and color impurities) that may prevent achieving the federally required cold soak filtration specification. Upon passing through the ion exchange resin process 40, the alkyl esters, which at this stage may be referred to as biodiesel, may be stored in product tanks (not shown).

Thus, the present invention provides a method and system for the production of biodiesel from high-FFA feedstock, such as used cooking oils, brown grease, trap grease, acidulated soaps, and byproducts of vegetable oil refining. These feedstocks are attractive raw materials for biodiesel production because they are typically waste byproducts from other processes, such as cooking and producing refined oils, and are therefore relatively inexpensive. However, such feedstock was previously unusable for the production of biodiesel because FFA content above approximately 4% inhibits or interferes with chemical catalysts used in the transesterification reaction. To solve this problem, the present invention includes the use of liquid enzymatic catalysts, which allows for the use of feedstocks having up to 100% FFA.

The liquid enzymatic catalysts (that is, enzymes) used in the present invention allow for the use of high-FFA feedstock. To make the process cost effective, the present invention also provides a means for recovering the enzymes from the heavy phase for reuse in subsequent reactions. For example, this means may be a membrane separation device that efficiently separates the enzymes from the crude glycerol.

Additionally, the high level of FFA in the feedstock may result in a relatively high level of FFA in the light phase, which must be neutralized and removed in order for the biodiesel product to meet quality specifications. Unlike currently known biodiesel production methods and systems, the present invention accomplishes this in a cost-effective manner without the need for harmful chemicals, high levels of energy, heat, and pressure. Specifically, the present invention uniquely includes ultrasonic cavitation of the light phase. The use of ultrasonic cavitation when applied to the light phase from the enzymatic process, in conjunction with the appropriate chemicals being added, may produce a reduction of the FFA to acceptable levels, reduction in color contaminants left over from the original feedstock, removal of polymeric contaminants, and the improved reduction in monoglyceride levels.

Although use of ultrasonic cavitation addresses the limitations of currently known methods, the alkyl esters produced from the ultrasonic reaction process may still require final treatment to meet required quality specifications. Therefore, the present invention addresses this need by including ion exchange technology to purify the alkyl esters as the final stage of the process. Although processes such as enzyme catalysis, ultrasonic cavitation, and ion exchange are known technologies, they have not been used together for the production of biodiesel from high-FFA feedstock. In fact, the use of these technologies together inventively addresses all of the limitations of these technologies when used individually.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of producing alkyl esters, the method comprising:
processing a high free fatty acid feedstock including a mixture of triglycerides and free fatty acids to remove water and solids to create a processed feedstock;
introducing and mixing the processed feedstock into a reaction vessel, the reaction vessel including water, at least one enzyme, and alcohol;
separating the reacted contents into a glycerin phase and an alkyl ester phase; and
treating the alkyl ester phase with a primary alcohol and a flocculant.

2. The method of claim 1, wherein the at least one enzyme is retained in the reaction vessel for reuse in subsequent reactions.

3. The method of claim 1, wherein treating the alkyl ester phase further includes washing the alkyl ester phase with an aqueous base before being treated with methanol and a flocculant.

4. The method of claim 1, wherein the flocculant is a polyacrylamide cationic polymer.

5. The method of claim 1, wherein the alkyl ester phase has a weight, and the percentage of methanol used to treat the alky ester phase is approximately 2 percent of the weight of the alkyl ester phase.

6. The method of claim 1, wherein at least a portion of the free fatty acids are recycled and used in feedstocks in subsequent reactions to increase overall yield of alkyl esters.

7. The method of claim 1, further including recycling the at least one enzyme from the reaction vessel.

8. The method of claim 1, further including converting the free fatty acids to alkyl esters after separation of the alkyl ester phase form the glycerin phase.

9. The method of claim 8, wherein converting the free fatty acids to alkyl esters includes treating the alkyl ester phase with an alcohol and a catalyst.

10. The method of claim 1, further including removing alcohol and water from the alkyl ester phase.

11. The method of claim 1, further including passing the alkyl ester phase through an ion exchange dry wash resin.

12. A method of producing alkyl esters, the method comprising:
processing a high free fatty acid feedstock including a mixture of triglycerides and free fatty acids to remove water and solids to create a processed feedstock;
introducing and mixing the processed feedstock into a reaction vessel, the reaction vessel including water, at least one enzyme, and alcohol;
separating the reacted contents into a glycerin phase and an alkyl ester phase; and
washing the alkyl ester phase with an aqueous base; and
treating the alkyl ester phase with a methanol and a polyacrylamide cationic polymer.

13. The method of claim 12, further including removing alcohol and water from the alkyl ester phase by vacuum distillation.

14. The method of claim 13, further including passing the alkyl ester phase through an ion exchange dry wash.

15. The method of claim 12, wherein the alkyl ester phase has a weight, and the percentage of methanol used to treat the alky ester phase is approximately 2 percent of the weight of the alkyl ester phase.

16. The method of claim 12, wherein the at least one enzyme is retained in the reaction vessel for reuse in subsequent reactions.

17. The method of claim 12, further including converting the free fatty acids to alkyl esters after separation of the alkyl ester phase form the glycerin phase.

18. The method of claim 17, wherein converting the free fatty acids to alkyl esters includes treating the alkyl ester phase with an alcohol and a catalyst.

19. The method of claim 12, further including recycling the at least one enzyme from the reaction vessel.

20. A method of producing alkyl esters, the method comprising:
processing a high free fatty acid feedstock including a mixture of triglycerides and free fatty acids to remove water and solids to create a processed feedstock;
introducing and mixing the processed feedstock into a reaction vessel, the reaction vessel including water, at least one enzyme, and alcohol;
separating the reacted contents into a glycerin phase and an alkyl ester phase; and
recycling a portion of the at least one enzyme from the glycerin phase;
removing water and the least one alcohol from the alkyl ester phase;
converting free fatty acids in the alkyl ester phase into alkyl esters;
washing the alkyl ester phase with an aqueous base; and
treating the alkyl ester phase with a methanol and a polyacrylamide cationic polymer, wherein the alkyl ester phase has a weight, and the percentage of methanol used to treat the alky ester phase is approximately 2 percent of the weight of the alkyl ester phase.
